(19) United States
(12) Patent Application Publication
(10) Pub. No.: US 2012/0029245 A1
(43) Pub. Date: Feb. 2, 2012

(54) CATALYTIC REACTIONS USING IONIC LIQUIDS

(75) Inventors: Andrew Corradini, Foster City, CA (US); Jarod McComick, Menlo Park, CA (US)

(73) Assignee: Oberon Fuels, Inc., La Jolla, CA (US)

(21) Appl. No.: 13/256,927

(22) PCT Filed: Mar. 17, 2010
(86) PCT No.: PCT/US10/27681

§ 371 Date: Oct. 13, 2011

Related U.S. Application Data
(60) Provisional application No. 61/161,017, filed on Mar. 17, 2009.

(51) Int. Cl. C07C 27/00; C07C 1/20

(52) U.S. Cl. 568/671; 568/840; 585/733

(57) ABSTRACT

A method of catalytically forming reaction products can include providing an ionic liquid phase. The ionic liquid phase can include a catalyst and an ionic liquid. At least one reactant can be reacted (14,16) in the ionic liquid phase to produce reaction products which include at least one of methanol and dimethyl ether. In particular, the ionic liquid can be hydrophobic sufficient to prevent substantial water in the ionic liquid phase. The use of ionic liquids can substantially reduce or eliminate residual catalyst carrier, i.e. ionic liquid, in downstream steps.

### RELATED APPLICATION

This application claims the benefit of U.S. Provisional Patent Application No. 61/161,017, filed Mar. 17, 2009 and which is incorporated herein by reference.

### BACKGROUND OF THE INVENTION

Modern society demands substantial energy and fuel to supply both essential needs and consumer wants. Conventional petroleum and fuel sources have proven to be a volatile resource in terms of international energy dependencies, real and perceived environmental issues, and an unknown limited supply. Alternative sources of suitable fuels has led to a wide variety of efforts such as corn to ethanol processes, biomass to liquid processes, algae to biodiesel processes, and a number of methane conversion processes. Methane to gasoline processes such as the Mobil process, Fischer-Tropsch process, and the like have seen commercial use. However, these processes can be difficult to control and often suffer from catalyst deactivation. These processes are also only economical at very large volume scales which require large initial capital investments. Each of these and other current alternatives have both benefits and drawbacks.

Furthermore, production of a wide range of chemical products requires the use of catalysts. However, the tailorability of these processes is limited and can require frequent catalyst replacement and/or reactivation.

### BRIEF DESCRIPTION OF THE DRAWINGS

The present invention will become more fully apparent from the following description and appended claims, taken in conjunction with the accompanying drawings. Understanding that these drawings merely depict exemplary embodiments of the present invention and they are, therefore, not to be considered limiting of its scope. It will be readily appreciated that the components of the present invention, as generally described and illustrated in the figures herein, could be arranged, sized, and designed in a wide variety of different configurations. Nonetheless, the invention will be described and explained with additional specificity and detail through the use of the accompanying drawings in which:

FIG. 1 is a flow diagram of a process for producing hydrocarbon fuels in accordance with one aspect.

### DETAILED DESCRIPTION

The following detailed description of exemplary embodiments of the invention makes reference to the accompanying drawings, which form a part hereof and in which are shown, by way of illustration, exemplary embodiments in which the invention may be practiced. While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

The following detailed description and exemplary embodiments of the invention will be best understood by reference to the accompanying drawings, wherein the elements and features of the invention are designated by numerals throughout.

### DEFINITIONS

In describing and claiming the present invention, the following terminology will be used.

The singular forms “a,” “an,” and “the” include plural referents unless the context clearly dictates otherwise. Thus, for example, reference to “a catalyst” includes reference to one or more of such materials and reference to “reacting” refers to one or more such steps. Furthermore, unless explicitly stated otherwise, reaction steps can be performed sequentially and/or in parallel and can be performed in a common vessel or separate vessels.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Processes for Forming Methanol and/or Dimethyl Ether

A method of catalytically forming reaction products can include providing an ionic liquid phase. The ionic liquid phase can include a catalyst and an ionic liquid. At least one reactant can be reacted in the ionic liquid phase to produce the reaction products. In particular, the ionic liquid can be hydrophobic sufficient to prevent substantial water in the ionic liquid phase and subsequent catalyst deactivation. The use of ionic liquids can substantially reduce or eliminate residual catalyst carrier, i.e. ionic liquid, in downstream steps. In particular, the extremely low vapor pressures of ionic liquids can allow for nearly complete separation of reaction products and unreacted reactants from the ionic liquid phase via simple separations, e.g. single stage distillation. Thus, ionic liquids are not inert in that the ions maintain low vapor pressures and prevent liquid phase elutriation.

Suitable catalysts can depend on the particular reaction. However, in one aspect, the catalyst is a particulate solid catalyst. Alternatively, the catalyst can be a liquid which is at least partially or fully miscible in the ionic liquid. Non-limiting examples of suitable catalysts can include methanol/dimethyl ether catalysts such as γ-alumina, Cu—Zn-alumina, Cu—ZnO—MnO, Cu—Al—Zn, CuAl₂, ZSM, SiO₂—Al₂O₃, Fe-based, Co-based, Ru— based, composites thereof, and combinations thereof. In one aspect, the catalysts include at least one of as γ-alumina and Cu—Zn-alumina. Most commercially available catalysts require activation (e.g. reduction) prior to use. Therefore, it can be desirable to activate the catalyst in situ. For example, U.S. Pat. No. 4,801,574, which is incorporated herein by reference, describes one such approach for activating a catalyst in situ of a reaction vessel or system. This basic approach can be applied to many of the catalysts described herein. Another approach is to inject a reducing agent, e.g. dilute hydrogen, into the reaction along with, or prior to, reacting the reactants. This can be accomplished, for example, by stoichiometric activation with hydrogen in a carrier gas. Non-limiting examples of carrier gases can include nitrogen, argon and the like. Typically, the hydrogen is present at less than about 10 volume percent.

Ionic liquids include an anion and a cation having a relatively low melting point, e.g. room-temperature ionic liquids. Further, analogous liquids such as deep eutectic solvents can also be suitable. The particular choice of each can determine the ionic liquid properties and provides significant tailorability to match particular reactants and/or reaction conditions. In particular, it can be desirable to adjust solubility of water and/or other reaction products in the liquid phase in order to prevent catalyst deactivation and to facilitate separation of products or reduce undesirable by-products. For example, hydrophobicity of the ionic liquid can be tuned by substituting H terminated alkyl chain groups, such as ethyl and isopropyl, with F terminated alkyl chain groups. In one aspect, the ionic liquid can be a liquid at 150-300° C., although the same ionic liquids may be a solid below about 150° C.

Non-limiting examples of suitable anions include halides such as CL⁻, Br⁻, and I⁻, [BF₄]⁻, [AlCl₄]⁻, [GaCl₄]⁻, [AuCl₃]⁻, [PF₆]⁻, [AsF₆]⁻, [NO₃]⁻, [NO₂]⁻, [CH₃CO₂]⁻, [SO₄].2H₂O²⁻, [CF₃SO₃]⁻, [CF₃CO₂]⁻, [N(SO₂CF₃)₂]⁻, [N(CN₂]⁻, [CB₁₁H₁₂]⁻, [CB₁₁H₆Cl₆]⁻, [CH₃CB₁₁H₁₁]⁻, [C₂H₅CB₁₁H₁₁]⁻, and combinations thereof. One particularly suitable anion is [N(SO₂CF₃)₂]⁻. Generally, the suitable anions can be, but are not limited to, halides, sulfates, nitrates, nitrites, acetates, trifluoromethansulfonates, heteropolyanions, combinations thereof, and the like.

Non-limiting examples of suitable cations include tetraalkylammonium ([NR₄]⁺), imidazolium cations such as EMIM, HMIM (1-hexyl-3-methylimidazolium), RMIM, PMIM, BMIM, EMMIM, and PMMIM, [PR4]+, [SR4]+,

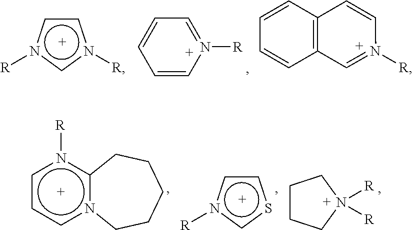

and combinations thereof. One particularly suitable cation is HMIM.

Other ionic liquids can include polyanionic liquids and polycationic liquids. Although the ionic liquid can often have a 1:1 anion to cation ratio, ionic liquids having a ratio of 2:1 (e.g. Gemini) or even 3:1 can be suitable and tend to have substantially lower vapor pressure than even those with 1:1 ratio. This is at least partially due to charge separation affects which require the ionic pairs/combinations for vaporization.

Selection of the anion can, in particular, affect hydrophobicity of the ionic liquid. For example, [N(SO₂CF₃)₂]⁻ (aka Tf2N), [PF₆]⁻, [BF₄]⁻, and the like tend to have strong hydrophobicity. Similarly, HMIM as the cation can generally provide substantial hydrophobicity. These highly hydrophobic ionic liquids can be particularly useful in preventing water from entering the ionic liquid phase. Water tends to oxidize and deactivate many catalysts such as those used in methanol and DME synthesis. On the other hand, EMIM tends to provide solubility for water thus reducing overall hydrophobicity of the ionic liquid.

Ionic liquids tend to have very good chemical and thermal stabilities. In some embodiments, a longer liquid-phase lifetime in the reactors can be achieved. Thus, spent catalyst can be removed from the ionic liquid phase, e.g. settling, filtration, etc. The ionic liquid can then be recycled and reused. The ionic liquids also provide decreased vapor pressure which can substantially reduce or eliminate slurry liquid elutriation. The above approaches can be applied in particular to formation of at least one of methanol and dimethyl ether. The reaction is thus a three phase system where ionic liquid, solid catalyst particle (non-dissolved), and gaseous reactants are present.

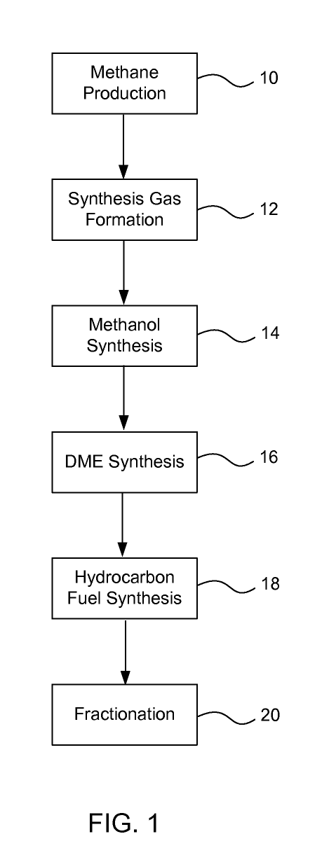

Referring now to FIG. 1, a process for producing a hydrocarbon fuel can begin by obtaining a hydrocarbon-containing gas in a methane production step 10. Although a methane-containing gas can often be productive, other hydrocarbon precursors, including without limitation C1-C4 hydrocarbons such as propane, butane, and ethane, may also be used. The hydrocarbon-containing gas can be synthesized or obtained from a suitable source. The hydrocarbon-containing gas can be produced in any of a number of processes which produce a methane-rich gas having a substantial proportion of carbon dioxide. Suitable processes can include, but are not limited to, anaerobic digestion, fungal decomposition of cellulosic or other plant matter (or, more generally, ‘biomass’), or other naturally occurring or man-made phenomena. The source gases for these processes can be from wastewater treatment, sewage treatment, septic tanks, natural gas, biomass conversion (analogous to composting), silage decomposition, or the like. In one specific aspect, the hydrocarbon-containing gas can be obtained by anaerobic digestion of organic constituents of municipal wastewater.

The digester off-gas or other hydrocarbon-containing gas can be optionally scrubbed in order to reduce impurities such as hydrogen sulfide and organics. Non-limiting examples of suitable scrubbing options can include zinc-oxide adsorbent, molybdenum-cobalt (Mo—Co) conversion of organic sulfur compounds to hydrogen sulfide, iron salt chemical treatment or iron sponge systems. Although actual ppm can range considerably, typical untreated digester gas can have about 200 ppm H₂S. In one specific embodiment, scrubbing the hydrocarbon-containing gas can be performed sufficient to remove substantially all H₂S.

Regardless of the source, the hydrocarbon-containing gas can generally have a majority of the hydrocarbon source, e.g. greater methane than any other single component. Although other ratios can be suitable, one embodiment includes about 60 vol % methane and about 40 vol % carbon dioxide. The range of methane may generally range from 50-70 vol % with the balance gas comprising or consisting essentially of carbon dioxide.

Synthesis gas (an industrially valuable mixture of hydrogen and carbon monoxide) can then be formed from the hydrocarbon-containing gas in a synthesis gas formation step 12. The process can include formation of methanol and/or dimethyl ether (DME). The methanol pathway can be followed by reacting the syn gas over the catalyst (e.g. CZA catalyst), to produce methanol. In the DME pathway, the syn gas can be reacted over a mixture of CZA and γ-alumina, for example. During this pathway, methanol is first formed over the CZA and then is subsequently dehydrated over the γ-alumina to form DME and water. This is where the hydrophobicity of the ionic liquid will become a more prominent factor. The methanol synthesis reaction creates only a small amount of water. The DME pathway creates much more water which is harder on the catalyst integrity. One specific embodiment includes reforming of the hydrocarbon-containing gas with steam to form syngas; other embodiments include without limitation partial oxidation and auto-thermal reforming. The inlet gases can be controlled to produce a synthesis gas having a H₂/CO ratio from 0.4 to 1.6 and from about 5 to about 10 vol % CO₂. Although not always required, the steam gas can further include oxygen and/or air. Optionally, a small amount of ambient air can be pulled into the reactor sufficient to balance the heat load. These ratios can be adjusted to balance the heat load in the reactor as well as provide the correct ratio of CO:H2:CO₂. The air can primarily be adjusted to stabilize temperature, and the water content can be used to decrease the amount of CO₂ and increase the H₂:CO ratio.

Specific operating parameters can be adjusted, however as a general guideline the steam reforming can be performed from about 750° C. to about 850° C. and about 0.5 psig to about 30 psig, such as about 800° C. and about 1 psig. Although results can vary, these conditions typically result in about 90% conversion efficiency of methane to carbon monoxide. The steam reforming can be accomplished using a reactor, although any device which allows for sufficient gas to catalyst contact surface area can be used.

In one aspect, a three-phase, slurry bubble column reactor can be used which bubbles the syn gas mixture through the ionic liquid. The bubbles can be created through any media which sparges or otherwise divides the gas mixture into discrete bubbles. Non-limiting examples of such media can include porous metal (e.g. stainless steel, Inconel, etc) or ceramic media (e.g. alumina, etc) or other similar media. The porous media can be a wire mesh, perforated plate or membrane, slotted layer, or other aperture layer. The size of the bubbles can generally range from 1 micron up to 10 cm, depending on the reactor design.

The reactor can optionally be kept isothermal. In one optional aspect, heat exchanger tubes can be contained within the reactor to control heat transfer. Alternatively, an external jacket or tubes can be used. In one specific embodiment, the tubes can contain water at a pressure that raises the boiling point to between 200-300° C. This can allow creation of steam and easily maintain the reaction at one specific temperature. However, other approaches can be used (e.g. process control feedback loops, synthetic heat transfer fluids, etc.).

In yet another aspect, the reactor can optionally have suitable freeboard space above the liquid height for gas disengagement from the liquid phase. This allows fluid to remain within the bed. Further, this approach provides a physical means of separation between the gas and liquid phases without additional downstream equipment. If a disengagement zone is designed having sufficient free space, then a negligible amount of the liquid phase will be allowed to exit the reaction zone.

The steam reforming can typically include a suitable catalyst such as, but not limited to, nickel, iridium, Ru, Rh, Pt, Pd, Co, Fe, Ag, or the like, and combinations or alloys thereof. These catalysts can be unsupported or supported on materials such as γ-alumina, calcium aluminate, regular amorphous alumina, lanthanum oxide, lanthanum aluminate, cesium oxide and specifically, other rare earth metal oxides and can include additives such as rare earth oxides, calcium oxides, and the like. In one specific embodiment, the catalyst can be an alumina-supported catalyst such as a Ni on alumina catalyst. More specifically, the nickel content can be from about 1 wt % to about 10 wt %, such as about 3 wt %.

For example, a 3% nickel catalyst can be produced by the incipient wetness technique. A 3% Ni content Ni(NO₃)₂ solution in water is first prepared. The amount of water is determined by the weight of the catalyst. Only enough water is used so that the catalyst will substantially completely absorb all of the solution. After the catalyst soaks up the solution, can be dried in ambient at 900° C. for 10 hours. Before use, the catalyst can be formed in 5% hydrogen balance nitrogen, forming gas at 500° C. for at least 1 hour. Alternatively, the alumina catalyst can be an Ir on alumina catalyst. Generally, the iridium content can be from about 0.5 to about 3 wt %, such as about 1 wt %.

The resulting synthesis gas product can be at least partially converted to a methanol product in a methanol synthesis step 14. This methanol synthesis step can generally involve a catalytic reaction. Furthermore, this step can utilize or be based on any number of methanol conversion processes such as, but not limited to, ICI low pressure methanol process, Katalco low pressure methanol process, Lurgi low pressure methanol process, Haldor-Topsoe process, liquid process such as the LPMeOH process, and the like. Suitable catalysts can include copper, zinc oxide, alumina, chromium oxide, and combinations thereof. In one aspect, the catalyst can be a zeolite catalyst or mixture of zeolite catalysts. In one specific embodiment, the catalytic reaction includes a Cu—Zn-Alumina (CZA) as a catalyst. Particle size of the catalyst can affect available surface area and catalytic activity. Therefore, in one aspect, the methanol synthesis catalyst can have an average particle size of about 20 μm to about 50 μm, although larger particle sizes can be used depending on scaling factors such as space-velocity/pressure drop optimization and the like. The CZA catalyst is typically provided commercially at about 4-8 mm in size. This larger size can be milled to the smaller more suitable (0.1-200 micron) sizes by ball milling, grinding or other suitable technique. Generally, suitable catalysts allow for the reactions to be primarily reaction rate limited rather than diffusion or mass transfer limited. In one specific embodiment, the catalyst further includes γ-alumina. For example, a particulate mixture can be formed of CZA and γ-alumina. Although conditions can vary, the catalytic process can often be performed at a temperature of about 200° C. to about 300° C., and in one embodiment from about 230° C. to about 240° C. The pressure can also be varied but is often from about 400 psig to about 1000 psig, such as about 600 psig. This methanol synthesis step is typically limited to about 10% conversion of CO to methanol. Thus, the product stream can be optionally recycled either with or without prior removal of the methanol product in order to achieve higher conversion.

Generally, the methanol product can be converted to the desired hydrocarbon fuel. This can be accomplished by partially converting the methanol product to a dimethyl ether product to form a mixture of methanol and dimethyl ether in a DME synthesis step 16. Optionally, the methanol synthesis from synthesis gas and the DME can be formed concurrently in a single step. The DME synthesis can involve a suitable DME catalyst such as, but not limited to, γ-alumina, Cu—Zn-alumina, H-ZSM-5, and combinations thereof. In one specific embodiment, the DME catalyst can consist essentially of γ-alumina and Cu—Zn-alumina catalyst particles, where the γ-alumina is about 5 to about 10 wt % of the DME catalyst. The DME catalyst can be supported or unsupported. In a particulate form, the DME catalyst can generally have a particulate size from about 1 micron to about 1000 micron, and typically from about 10 micron to about 100 micron. The resulting methanol-DME mixture can generally comprise from about 5 vol % to about 50 vol % methanol, and often from about 5% to about 10%, with the remainder being DME and typically a small portion of water.

The mixture of methanol and dimethyl ether can be converted to hydrocarbon fuel in a hydrocarbon fuel synthesis step 18. The mixture can be exposed to a ZSM catalyst under conditions sufficient to form the hydrocarbon fuel. The ZSM catalyst can be ZSM-5 having a silicon to aluminum ratio of about 24 to about 30. The catalyst can be supported or unsupported. Furthermore, the catalyst can often have a particle size of about 1 μm. Although conditions can vary, a general guideline for the formation of hydrocarbon fuel is to have a temperature from about 300° C. to about 400° C. and relatively low pressures, e.g. typically about 2 atm up to about 30 atm. Other suitable catalysts may also be used such as, but not limited to, ZSM-11, ZSM-12, ZSM-21, TEA mordenite and the like. The hydrocarbon fuel can vary somewhat in composition, but is often a gasoline mixture of aliphatic hydrocarbons having C5 to C12 chains and aromatic hydrocarbons including xylenes, toluenes, isopentene, and other isoparaffins.

The unrefined hydrocarbon fuel can be used, transported or stored as is, or may be further refined. For example, the hydrocarbon fuel can be fractionated into at least two fractions including light hydrocarbons and heavy hydrocabons in the conventional manner. The heavy fraction can generally include significant portions of durene which can be used or further converted to isodurene.

Each of the synthesis gas formation, methanol synthesis, DME synthesis, and hydrocarbon fuel synthesis steps can generally be performed in separate reactors. However, two or more of these steps can also be performed in a single reactor either sequentially or simultaneously.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A method of catalytically forming reaction products, comprising:
a) providing an ionic liquid phase including a catalyst and an ionic liquid;
b) reacting at least one reactant in the ionic liquid phase to produce the reaction products, said ionic liquid being hydrophobic sufficient to prevent substantial water in the ionic liquid phase and said reaction products including at least one of methanol and dimethyl ether.

2. The method of claim 1, wherein the catalyst is a particulate solid catalyst.

3. The method of claim 2, wherein the catalyst comprises or consists essentially of a methanol dimethyl ether catalyst selected from the group consisting of γ-alumina, Cu—Zn-alumina, Cu—ZnO—MnO, Cu—Al—Zn, CuAl₂, ZSM, SiO₂—Al₂O₃, Fe-based, Co-based, Ru-based, composites thereof, and combinations thereof.

4. The method of claim 3, wherein the catalyst includes at least one of γ-alumina and Cu—Zn-alumina.

5. The method of claim 1, further comprising activating the catalyst in situ by exposure to a dilute hydrogen gas.

6. The method of claim 1, wherein the ionic liquid includes an anion selected from the group consisting of halides, sulfates, nitrates, nitrites, acetates, trifluoromethansulfonates, heteropolyanions, combinations thereof.

7. The method of claim 6, wherein the anion is [N(SO₂CF₃)₂]⁻.

8. The method of claim 1, wherein the ionic liquid includes a cation selected from the group consisting of tetraalkylammonium ([NR₄]⁺, imidazolium, EMIM, HMIM (1-hexyl-3-methylimidazolium), RMIM, PMIM, BMIM, EMMIM, PMMIM, [PR4]+, [SR4]+, 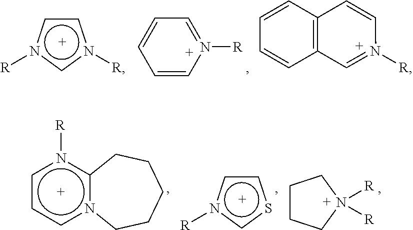
and combinations thereof.

9. The method of claim 8, wherein the cation is HMIM.

10. The method of claim 1, wherein the ionic liquid is a polyanionic liquid.

11. The method of claim 1, wherein the ionic liquid is a polycationic liquid.

12. The method of claim 1, wherein hydrophobicity of the ionic liquid is tuned by substituting H terminated alkyl chain groups with F terminated alkyl chain groups.

13. The method of claim 1, wherein ionic liquid is a solid at room temperature.

14. The method of claim 13, wherein the ionic liquid is a liquid at 150-300° C.

15. The method of claim 1, wherein the reaction products include methanol, and the method further comprises partially converting the methanol to dimethyl ether to form a mixture of methanol and dimethyl ether simultaneously with production of the methanol.

16. The method of claim 15, wherein the partially converting the methanol includes a DME catalyst consisting essentially of γ-alumina and Cu—Zn-alumina catalyst particles.

17. The method of claim 15, further comprising exposing the mixture of methanol and dimethyl ether to a zeolite catalyst under conditions sufficient to form the hydrocarbon fuel.
